(19) United States
(12) Patent Application Publication
(10) Pub. No.: US 2015/0133492 A1
(43) Pub. Date: May 14, 2015

(54) N-desmethyldauricine, A Novel Autophagic Enhancer for Treatment of Cancers and Neurodegenerative Conditions Thereof

(71) Applicant: Macau University of Science and Technology, Macau (CN)

(72) Inventors: Kam Wai WONG, Macau (CN); Yuen Kwan LAW, Macau (CN); Zhi Hong JIANG, Macau (CN); Liang LIU, Macau (CN); Wai Kit CHAN, Macau (CN); Xiao Jun YAO, Macau (CN)

(73) Assignee: Macau University of Science and Technology

(21) Appl. No.: 14/526,539

(22) Filed: Oct. 29, 2014

Related U.S. Application Data
(60) Provisional application No. 61/903,976, filed on Nov. 14, 2013.

(51) Int. Cl. A61K 31/4725

(52) U.S. Cl. CPC A61K 31/4725
USPC 514/308

(57) ABSTRACT

This invention is directed to the use of N-desmethyldauricine, a novel autophagy enhancer, in treating cancers or neurodegenerative conditions.

### CROSS-REFERENCE TO RELATED APPLICATION

This application claims benefit under 35 U.S.C. §119(e) of U.S. Provisional Application having Ser. No. 61/903,976 filed 14 Nov. 2013, which is hereby incorporated by reference herein in its entirety.

### FIELD OF INVENTION

This invention relates to a novel autophagy enhancer and the use thereof in treating cancers and neurodegenerative conditions. In particular, the autophagy enhancer is isolated from Chinese traditional medicine.

### BACKGROUND OF INVENTION

Autophagy is a cellular degradation process that involves the delivery of cytoplasmic cargo such as long-lived protein, mis-folded protein or damaged organelles, sequestered inside double-membrane vesicles to the lysosome. Autophagy occurs at low basal levels in cells to maintain normal homeostatic functions by protein and organelle turnover. Upon cellular stressful conditions such as nutrient deprivation, oxidative stress, infection or protein aggregate accumulation, autophagy starts with membrane isolation and expansion to form the double-membraned vesicle (autophagosome) that sequesters the cytoplasmic materials. Followed by fusion of the autophagosome with lysosome to form an autolysosome, all the engulfed materials are degraded to recycle intracellular nutrients and energy¹. Impaired autophagy and the age-related decline of this pathway favour the pathogenesis of many diseases that occur especially at higher age such as cancers and neurodegenerative diseases².

One of the key roles for autophagy is to degrade toxic aggregate-prone cytoplasmic proteins that are inaccessible to the proteasome when they form oligomers or aggregates³, aggregate-prone proteins with polyglutamine and polyalanine expansions, in turn, are degraded by autophagy⁴. Inhibition of mTOR induces autophagy and reduces toxicity of polyglutamine expansions in fly and mouse models of Huntington disease^{5,6}. These proteins include mutant α-synuclein which causes Parkinson's disease, and polyglutamine-expanded mutant huntingtin that causes Huntington's disease^{7,8}. Autophagy induction reduces mutant huntingtin levels and protects against its toxicity in cells, D. melanogaster and mouse models ^{4,5}. Similar effects are observed in polyQ-containing cells and fly models⁹. In contrast, protein aggregates form in the cytoplasm when autophagy is inhibited in normal mice¹⁰. Rapamycin, a FDA-approved immunosuppressant, is found effective in treating fruit fly and mouse models of Huntington's disease through increased autophagic clearance of mutant huntingtin⁵. Besides, a small-molecule screen also revealed new chemicals that decrease mutant huntingtin toxicity through autophagy⁸.

While autophagy may play a protective role in neurodegenerative disease⁸, autophagic dysfunction is associated with DNA damage, chromosome instability^{11,12}, and increased incidence of malignancies¹². Modulators of autophagy may play a protective role through promoting autophagic cell death in tumors or augment the efficacy of chemotherapeutic agents when used in combination. Several clinically approved or experimental antitumor agents induced autophagy-related cell death¹³⁻¹⁶.

### SUMMARY OF INVENTION

In light of the foregoing background, it is an object of the present invention to provide a novel autophagy enhancer, N-desmethyldauricine, with its potential therapeutic application in cancers and neurodegenerative diseases by direct targeting SERCA protein, leading to induction of autophagy-related cell death in a panel of cancer cells and clearance of mutant huntingtin in neuronal cells.

Accordingly, the present invention, in one aspect, provides a method of treating cancer which includes administering a therapeutically effective amount of N-desmethyldauricine to a subject in need thereof.

In an exemplary embodiment, the cancer is cervical cancer, lung cancer, breast cancer, prostate cancer or liver cancer.

In another exemplary embodiment, N-desmethyldauricine selectively induces autophagic cell death in cancer cells or apoptosis-resistant cells via direct inhibition of SERCA.

In yet another aspect, the present invention provides a method of treating neurodegenerative disorder including administering a therapeutically effective amount of N-desmethyldauricine to a subject in need thereof.

In an exemplary embodiment, N-desmethyldauricine removes Huntingtin aggregates via autophagy induction and reduces the aggregate-mediated cell cytotoxicity in neuronal cells.

In a further exemplary embodiment, the neurodegenerative disease is Alzheimer's disease, Parkinson's disease, Huntington's disease, amyotrophic lateral sclerosis, ataxia telangiectasia, spinocerebellar atrophy or multiple sclerosis.

In another aspect, the present invention provides a method of inducing autophagic cell death selectively in apoptosis-resistant cells. The method comprises exposing the apoptosis-resistant cells to a composition comprising N-desmethyldauricine to induce autophagy via SERCA inhibition in the cells.

### BRIEF DESCRIPTION OF FIGURES

FIG. 1a shows the chemical structure of N-desmethyldauricine (LP-4).

FIG. 1b shows the results of cell cytotoxicity study of N-desmethyldauricine towards a panel of cancer and normal cells.

FIG. 2a and FIG. 2b show that N-desmethyldauricine induces autophagic GFP-LC3 puncta formation in HeLa cancer cells and a panel of cancer and normal cells by immunocytochemistry.

FIG. 3 shows that N-desmethyldauricine-induced autophagosome/autolysosome formation is visualized by electronic microscopy in HeLa cancer cells.

FIG. 4a shows that N-desmethyldauricine induces autophagic protein LC3 conversion from LC3-I to LC3-II in HeLa cancer cells.

FIG. 4b shows that N-desmethyldauricine induces autophagic flux in HeLa cancer cells.

FIG. 5a and FIG. 5b show that N-desmethyldauricine-induced autophagy is dependent on the presence of autophagy-related gene7 (Atg7).

FIG. 6a and FIG. 6b show the results of RT² Profiler™ PCR Array of N-desmethyldauricine.

FIG. 7a and FIG. 7b show that the genes PERK, Igf-1 and Ulk1 are further validated for their participation in N-desmethyldauricine (LP-4)-mediated autophagy induction.

FIG. 8 shows that N-desmethyldauricine (LP-4) activates autophagy through modulation of AMPK-mTOR signaling pathway.

FIG. 9a and FIG. 9b show that the suppression of AMPK, CaMKK-β and calcium chelation will abolish the N-desmethyldauricine (LP-4)-mediated autophagy and LC3-II conversion.

FIG. 10a and FIG. 10b show that N-desmethyldauricine mobilizes the cytosolic calcium level in HeLa cancer cells.

FIG. 11a shows a 3D schematic representation (ribbon diagram) illustrating N-desmethyldauricine binding and suppressing the SERCA pump and,

FIG. 11b shows percentage of Ca²⁺ ATPase activity of SERCA in the presence of N-desmethyldauricine.

FIG. 12a and FIG. 12b show that N-desmethyldauricine is able to induce autophagic cell death in wild-type Atg7 cells, but not in Atg7 deficient cells.

FIG. 13a, FIG. 13b and FIG. 13c show that N-desmethyldauricine is able to induce cell death in apoptosis-resistant cells.

FIG. 14a, FIG. 14b and FIG. 14c show the cell cytotoxicity, clearance of HTT mutant and reduction aggregates-mediated cytotoxic effect of N-desmethyldauricine.

### DETAILED DESCRIPTION OF THE PREFERRED EMBODIMENTS

As used herein and in the claims, “comprising” means including the following elements but not excluding others.

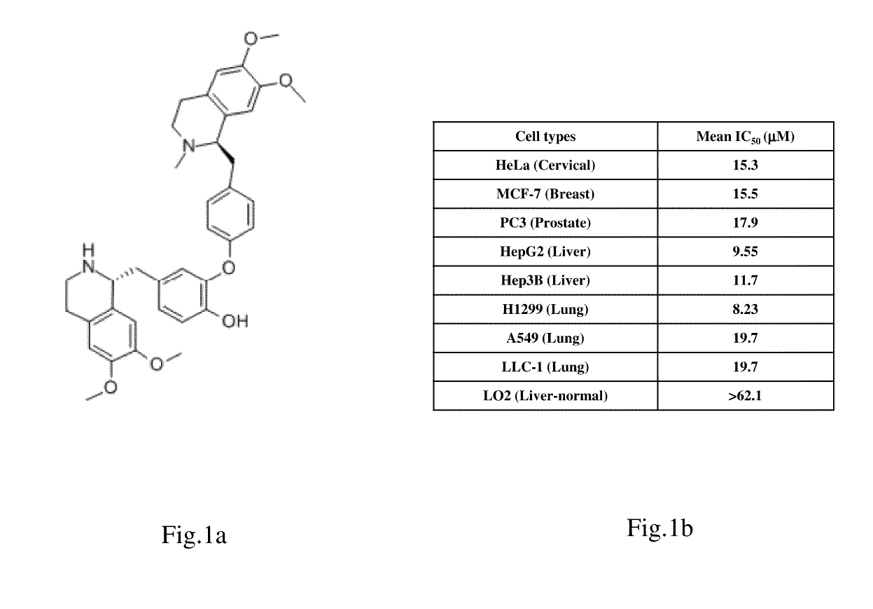

This invention provides the use of N-desmethyldauricine isolated from Chinese medicinal herbs, rhizoma of Menispermum dauricum DC, with chemical structure as shown in FIG. 1a, in treating cancers and neurodegenerative conditions.

The following preparations and examples are given to enable those skilled in the art to more clearly understand and to practice the present invention. They should not be considered as limiting the scope of the invention, but merely as being illustrative and representative thereof.

### Example 1

This example describes in vitro cell cytotoxicity of N-desmethyldauricine in a panel of human cancer and normal cells.

### Cell Culture and Cytotoxicity Assay

The test compound of N-desmethyldauricine was dissolved in DMSO at a final concentration of 100 mmol/L and stored at −20° C. Cytotoxicity was assessed using the 3-(4,5-dimethylthiazol-2-yl)-2,5-diphenyltetrazolium bromide assay as previously described¹⁷. Cell number, 4000-8000 of HeLa (human cervical cancer), MCF-7 (human breast cancer), HepG2 (human liver cancer), Hep3B (human liver cancer), H1299 (human lung cancer), A549 (human lung cancer), PC3 (human prostate cancer), LLC-1 (mouse Lewis lung carcinoma) and LO2 (human normal liver) cells were seeded on 96-well plates per well, respectively. After overnight pre-incubation, the cells were exposed to different concentrations of N-desmethyldauricine (namely 100, 50, 25, 12.5, 6.25, 3.125, 1.5625, 0.78, 0.39, 0.195, 0.079, 0.039 μmol) for 3 days. Subsequently, 10 μL of MTT reagents was added to each well and incubated at 37° C. for 4 hours followed by the addition of 100 μL solubilization buffer (10% SDS in 0.01 mol/L HCl) and overnight incubation. Absorbance at 585 nm was determined from each well on the following day. The percentage of cell viability was calculated using the following formula: Cell viability (%)=Cells number treated/Cells number DMSO control×100. Data was obtained from three independent experiments.

### Results

Significant cell cytotoxicity was observed with mean IC₅₀ value ranging from 8.23-19.7 μM observed in a panel of human cancer cells treated with N-desmethyldauricine for 72 hours as revealed by MTT assay as shown in FIG. 1b. However, N-desmethyldauricine indicated no or less cytotoxic effect toward human normal liver cells with IC₅₀>62.1 μM.

### Conclusion

N-desmethyldauricine exhibits potent and specific cell cytotoxicity toward a panel of human cancer cells, but not in normal human liver LO2 cells.

### Example 2

This example describes an in vitro study to demonstrate the autophagic effect of N-desmethyldauricine.

### Quantification of Autophagy GFP-LC3 Puncta

GFP-LC3 puncta formation was quantified as previously described¹⁵. In brief, cells grown on coverslips in a 6-well plate were treated with or without 10 μM of N-desmethyldauricine for 4 hours, the cells were then fixed in 4% paraformaldehyde for 20 minutes at room temperature and then rinsed with PBS. Slides were mounted with FluorSave™ mounting media (Calbiochem, San Diego, Calif.) and examined by fluorescence microscopy. The number of GFP-positive cells with GFP-LC3 puncta formation was examined under the Nikon ECLIPSE 80i microscope. Representative images were captured with CCD digital camera Spot RT3™ (Diagnostic Instruments, Inc., Melville, N.Y.). To quantify for autophagy, the percentage of cells with punctate GFP-LC3 fluorescence was calculated by counting the number of the cells with punctate GFP-LC3 fluorescence in GFP-positive cells. A minimum of 150 cells from 3 randomly selected fields was scored.

### Results

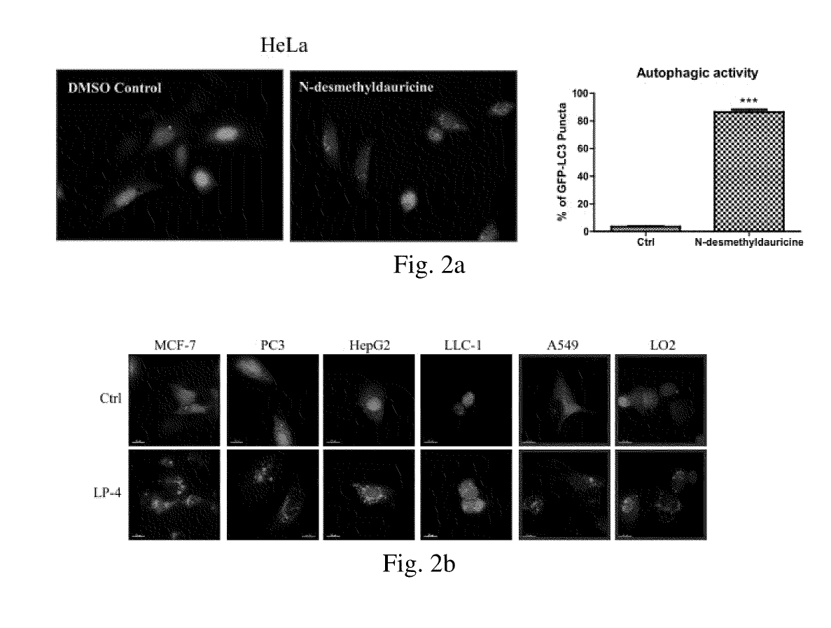

As compared to DMSO control treatment, N-desmethyldauricine significantly induced the GFP-LC3 puncta formation in HeLa cancer cells as shown FIGS. 2a. In addition, N-desmethyldauricine also increased the formation of GFP-LC3 puncta formation toward a panel of cancer and normal cells as revealed by fluorescent microscopy as shown in FIG. 2b.

### Conclusion

These data suggest that N-desmethyldauricine is a novel autophagy enhancer. Although N-desmethyldauricine could induce autophagy in LO2 human normal liver cells, but the N-desmethyldauricine-mediated autophagy exhibits no observable cytotoxic effect on human normal cells (as shown in FIG. 1b), suggesting the N-desmethyldauricine-mediated cytotoxic effect is tumor specific.

### Example 3

This example describes an in vitro study to visualize the N-desmethyldauricine—induced autophagosomes/autolysosomes by electronic microscopy.

### Transmission Electron Microscopy

N-desmethyldauricine treated HeLa cells were fixed overnight with 2.5% glutaraldehyde followed by a buffer wash. Samples were post-fixed in 1% OsO4 and embedded in Araldite 502. Ultrathin sections were double stained with uranyl acetate and lead citrate, and analyzed by Philips CM 100 transmission electron microscope at a voltage of 80 kV.

### Results

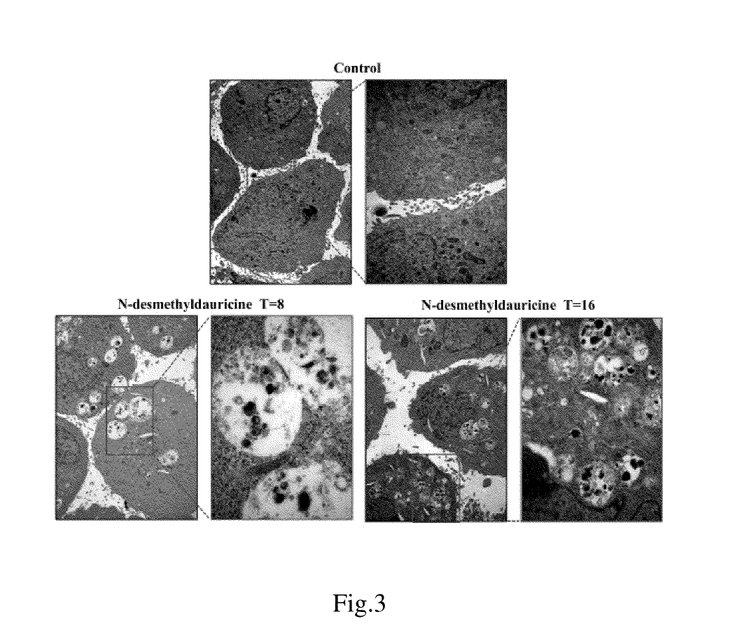

The autophagosomes/autolysosomes were found in N-desmethyldauricine treated HeLa cancer cells as shown in FIG. 3.

### Conclusion

These data suggest that N-desmethyldauricine is a novel autophagy enhancer and able to induce autophagosomes/autolysosomes in human cancer cells.

### Example 4

This example describes an in vitro study to demonstrate the autophagic marker protein conversion by N-desmethyldauricine.

### Detection of Autophagic Marker Protein LC3 Conversion

After N-desmethyldauricine treatments, HeLa cancer cells were harvested and lysed in RIPA buffer (Cell Signaling Technologies Inc. (Beverly, Mass.). The cell lysates were then resolved by SDS-PAGE. After electrophoresis, the proteins from SDS-PAGE were transferred to nitrocellulose membrane which was then blocked with 5% non-fat dried milk for 60 minutes. The membrane was then incubated with LC3 primary antibodies (1:1000) in TBST overnight at 4° C. After that, the membrane was further incubated with HRP-conjugated secondary antibodies for 60 minutes. Finally, protein bands were visualized by using the ECL Western Blotting Detection Reagents (Invitrogen, Paisley, Scotland, UK).

### Detection of Autophagic Flux by N-desmethyldauricine

After N-desmethyldauricine treatments in the presence or absence of lysosomal inhibitor 10·M of E64d/Pepstatin A, HeLa cancer cells were harvested and lysed in RIPA buffer (Cell Signaling Technologies Inc., Beverly, Mass.). The cell lysates were then resolved by SDS-PAGE. After electrophoresis, the proteins from SDS-PAGE were transferred to nitrocellulose membrane which was then blocked with 5% non-fat dried milk for 60 minutes. The membrane was then incubated with LC3 primary antibodies (1:1000) in TBST overnight at 4° C. After that, the membrane was further incubated with HRP-conjugated secondary antibodies for 60 minutes. Finally, protein bands were visualized by using the ECL Western Blotting Detection Reagents (Invitrogen, Paisley, Scotland, UK).

### Results

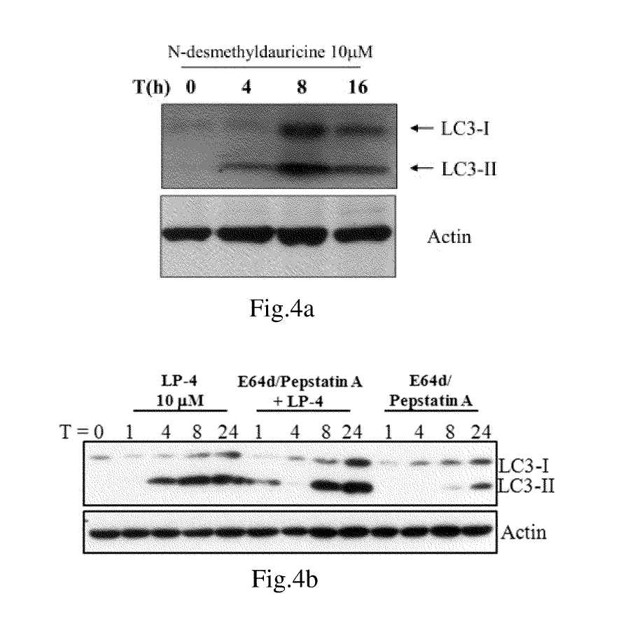

Western blot analysis showed that the autophagic marker LC3-II conversion was induced upon N-desmethyldauricine treatment as shown in FIG. 4a. In addition, N-desmethyldauricine was able to further enhance the LC3-II conversion in the presence of lysosomal inhibitor (E64d/Pepstatin A) as illustrated in FIG. 4b. Collectively, these data suggest that N-desmethyldauricine is able to induce autophagy via increasing of autophagic flux.

### Conclusion

These data suggest that N-desmethyldauricine is a novel autophagy enhancer.

### Example 5

This example describes an in vitro study to demonstrate the autophagic effect of N-desmethyldauricine is dependent on the presence of autophagy-related gene 7 (Atg7).

### Quantification of Autophagy GFP-LC3 Puncta in Atg7 Wild Type and Deficient MEFs

GFP-LC3 puncta formation was quantified as previously described¹⁵. In brief, both Atg7 wild-type and deficient mouse embryonic fibroblasts (MEFs) grown on coverslips in a 6-well plate were treated with indicated concentrations of N-desmethyldauricine. Both Atg7 wild-type and deficient mouse embryonic fibroblasts were then fixed in 4% paraformaldehyde for 20 minutes at room temperature and then rinsed with PBS. Slides were mounted with FluorSave™ mounting media (Calbiochem, San Diego, Calif.) and examined by fluorescence microscopy. The number of GFP-positive cells with GFP-LC3 puncta formation was examined under the Nikon ECLIPSE 80i microscope. Representative images were captured with CCD digital camera Spot RT3™ (Diagnostic Instruments, Inc., Melville, N.Y.). To quantify for autophagy, the percentage of cells with punctate GFP-LC3 fluorescence was calculated by counting the number of the cells with punctate GFP-LC3 fluorescence in GFP-positive cells. A minimum of 150 cells from 3 randomly selected fields was scored.

### Results

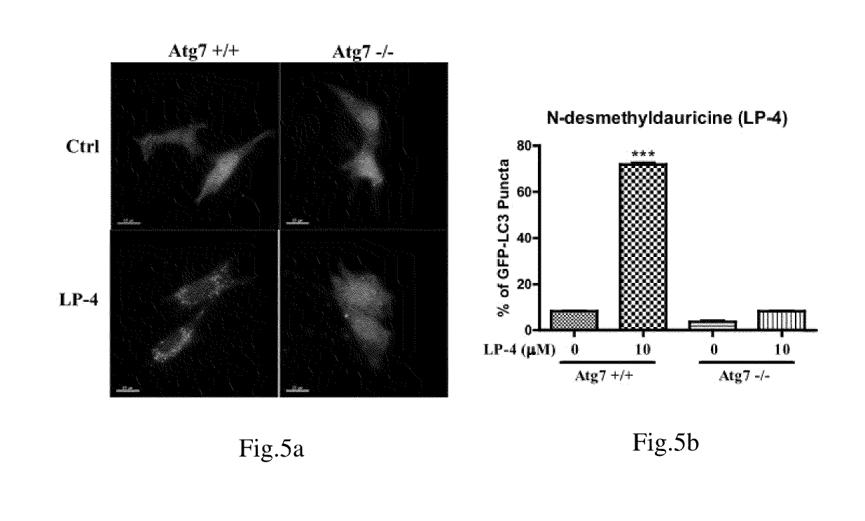

N-desmethyldauricine was found to induce GFP-LC3 puncta formation in wild type Atg7 cells (Atg7^{+/+}) but not in Atg7-knockout (Atg7^{-/-}) mouse embryonic fibroblasts as shown in FIG. 5a and 5b.

### Conclusion

N-desmethyldauricine works as a novel autophagy enhancer which depends on autophagy related gene, Atg7, for the induction of autophagy.

### Example 6

This example describes an in vitro study to demonstrate the gene regulation of N-desmethyldauricine during autophagy induction.

### RT² Profiler Autophagy PCR Array Analysis

For PCR array analysis, N-desmethyldauricine treated HeLa cells were used to obtain the total RNA by Qiagen RNeasy® Mini Kit (Qiagen). The autophagy pathway specific RT-PCR array was used to evaluate the potential alterations of related genes after N-desmethyldauricine treatments in HeLa cells. The autophagy array comprised 87 genes selected based on their involvement in regulating autophagy induction. There were 5 housekeeping genes served as positive controls. Total RNA was reverse transcripted using the RT2 First Strand Kit. Real-time PCR reactions were carried out on ABI 7500 (Applied Biosystems) using the RT2 SYBR® Green qPCR Mastermix (Qiagen) according to manufacturer's instructions. Data analysis was performed using the Qiagen's integrated web-based software package for the PCR Array System, which automatically performs all ΔΔCt based fold-change calculations from raw threshold cycle data.

### Quantification of Autophagy GFP-LC3 Puncta in the Presence of Gene Specific siRNA

GFP-LC3 puncta formation was quantified as previously described¹⁵. In brief, HeLa cells grown on coverslips in a 6-well plate were knockdown with control siRNA or PERK siRNA, IgF-1 siRNA and ULK-1 siRNA respectively, and then treated with 10 μM of N-desmethyldauricine for 4 hours, the cells were then fixed in 4% paraformaldehyde for 20 minutes at room temperature and then rinsed with PBS. Slides were mounted with FluorSave™ mounting media (Calbiochem, San Diego, Calif.) and examined by fluorescence microscopy. The number of GFP-positive cells with GFP-LC3 puncta formation was examined under the Nikon ECLIPSE 80i microscope. Representative images were captured with CCD digital camera Spot RT3™ (Diagnostic Instruments, Inc., Melville, N.Y.). To quantify for autophagy, the percentage of cells with punctate GFP-LC3 fluorescence was calculated by counting the number of the cells with punctate GFP-LC3 fluorescence in GFP-positive cells. A minimum of 150 cells from 3 randomly selected fields was scored.

### Results

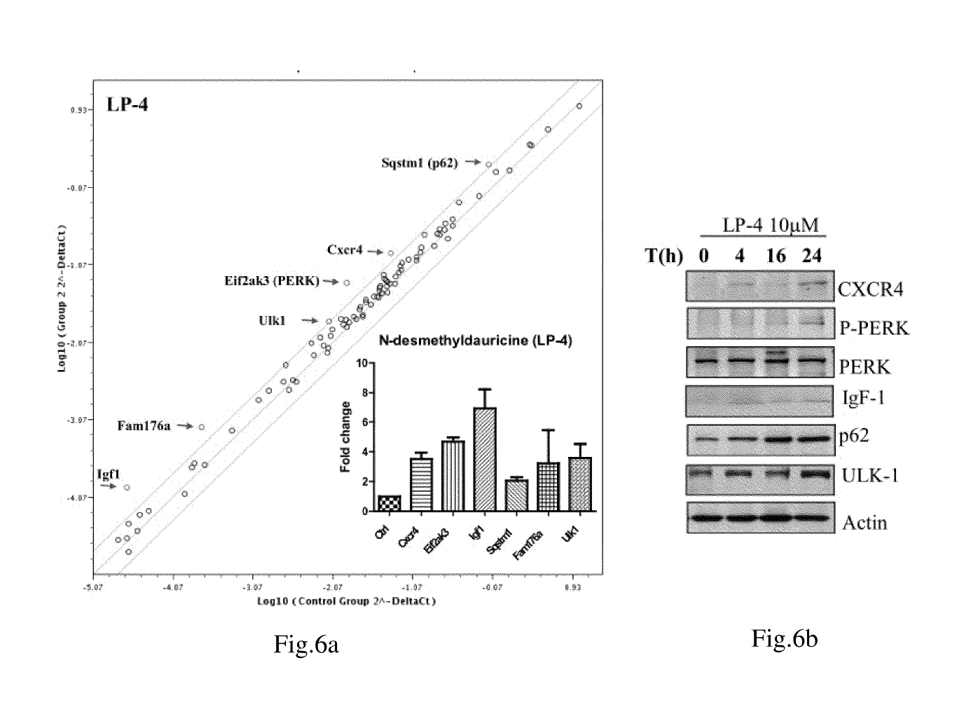

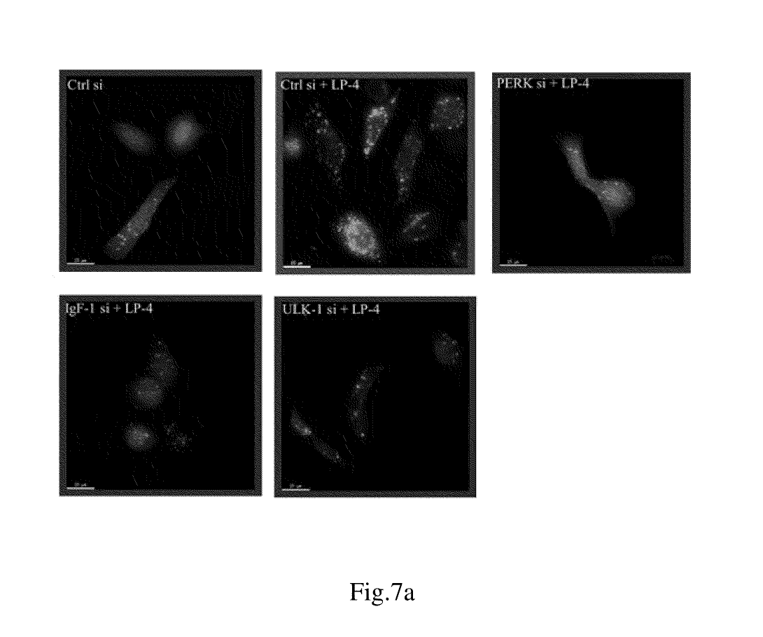

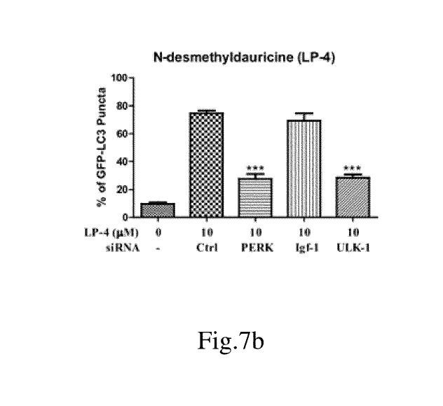

RT² Profiler™ PCR array analysis showed that N-desmethyldauricine (LP-4) induced autophagy through regulation of a panel of genes, i.e. Igf1, Fam176a, Ulk1, PERK, Cxcr4 and p62 as illustrated in FIG. 6a. Among this group of genes, Cxcr4, PERK, Igf-1, p62 and Ulk1 are validated by western blotting as shown in FIG. 6b. By siRNA gene knockdown method, the genes PERK, Ulk1 and Igf-1 are further confirmed to participate the N-desmethyldauricine (LP-4)-mediated autophagy induction (FIG. 7a and FIG. 7b).

### Conclusion

N-desmethyldauricine (LP-4) induces autophagy through regulation of genes, i.e. Ulk1 and PERK.

### Example 7

This example describes an in vitro study to demonstrate the mechanism and action of N-desmethyldauricine during autophagy induction.

### Detection of mTOR Signaling Marker Proteins

HeLa cells treated with indicated time and concentrations of N-desmethyldauricine were harvested and lysed in RIPA buffer (Cell Signaling). The cell lysates were then resolved by SDS-PAGE. After electrophoresis, the proteins from SDS-PAGE were transferred to nitrocellulose membrane which was then blocked with 5% non-fat dried milk for 60 minutes. The membrane was then incubated with P-p70S6K, p70S6K, P-AMPK, AMPK and actin primary antibodies (1:1000) in TBST overnight at 4° C. respectively. After that, the membrane was further incubated with HRP-conjugated secondary antibodies for 60 minutes. Finally, protein bands were visualized by using the ECL Western Blotting Detection Reagents (Invitrogen).

### Quantification of N-desmethyldauricine-Mediated Autophagy in the Presence of Specific Inhibitors

GFP-LC3 puncta formation was quantified as previously described¹⁵. In brief, HeLa cells expressing GFP-LC3 were treated with N-desmethyldauricine (LP-4, 10 μM) in the presence of AMPK inhibitor, compound C (CC, 10 μM), CaMKK-β inhibitor, STO-609 (25 μM) or Calcium chelator, BAPTA/AM (BM, 10 μM) for 4 hours. The cells were then fixed in 4% paraformaldehyde for 20 minutes at room temperature and then rinsed with PBS. Slides were mounted with FluorSave™ mounting media (Calbiochem) and examined by fluorescence microscopy. To quantify for autophagy, the percentage of cells with punctate GFP-LC3 fluorescence was calculated by counting the number of the cells with punctate GFP-LC3 fluorescence in GFP-positive cells. A minimum of 150 cells from 3 randomly selected fields was scored.

### Calcium Detection by Flow Cytometry Analysis

Changes in intracellular free calcium were measured by a fluorescent dye, Fluo-3, as described previously¹⁸. Briefly, HeLa cells were washed twice with MEM medium after N-desmethyldauricine (LP-4) treatment (5 μM/10 μM) for various times (1 h, 2 h, 4 h). Then cell suspensions were incubated with 5 μM Fluo-3 at 37° C. for 30 min. Then the cells were washed twice with HBSS. After re-suspended cell samples were subjected to FACS analysis, at least 10,000 events were analyzed.

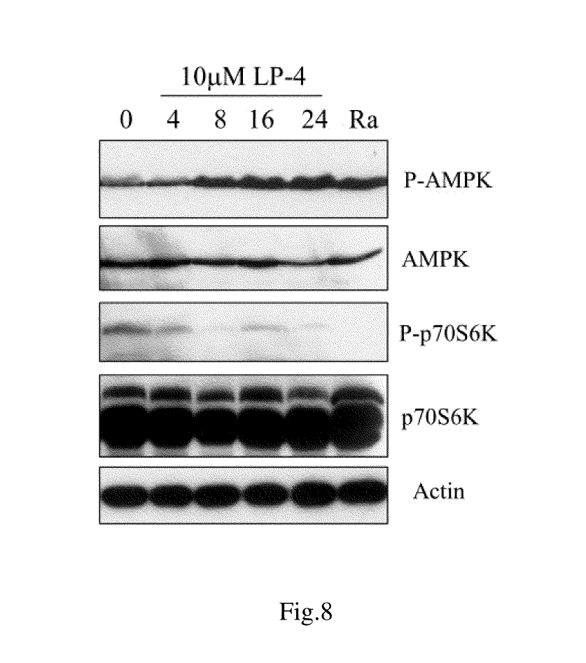

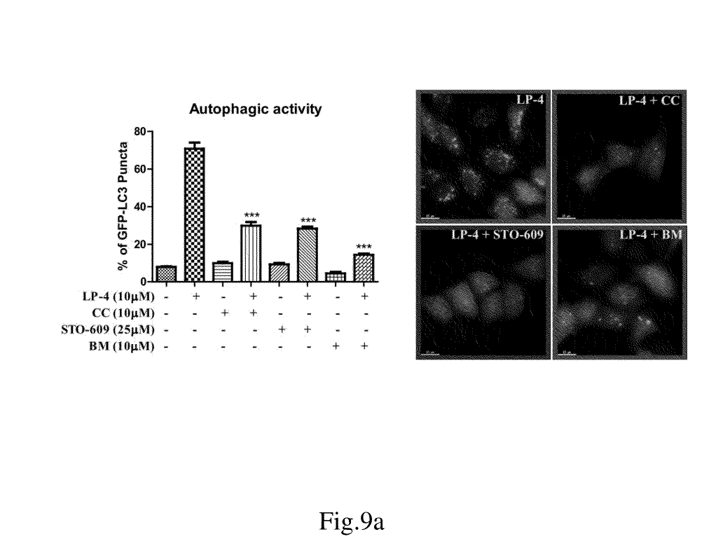

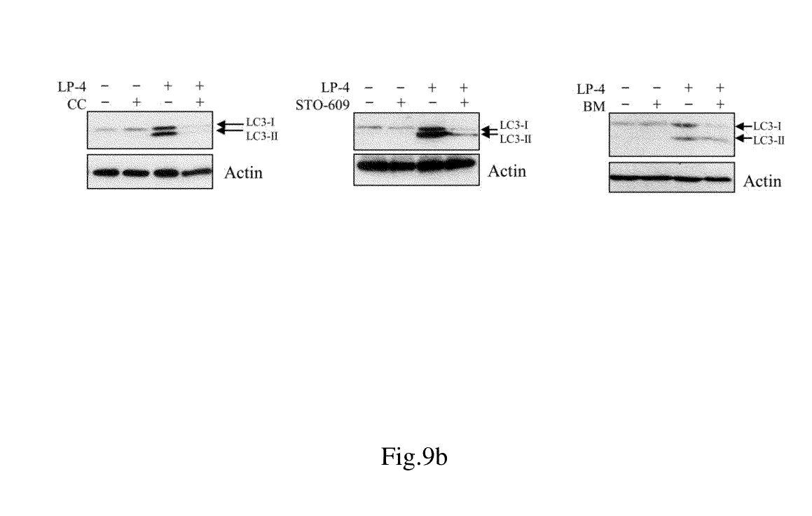

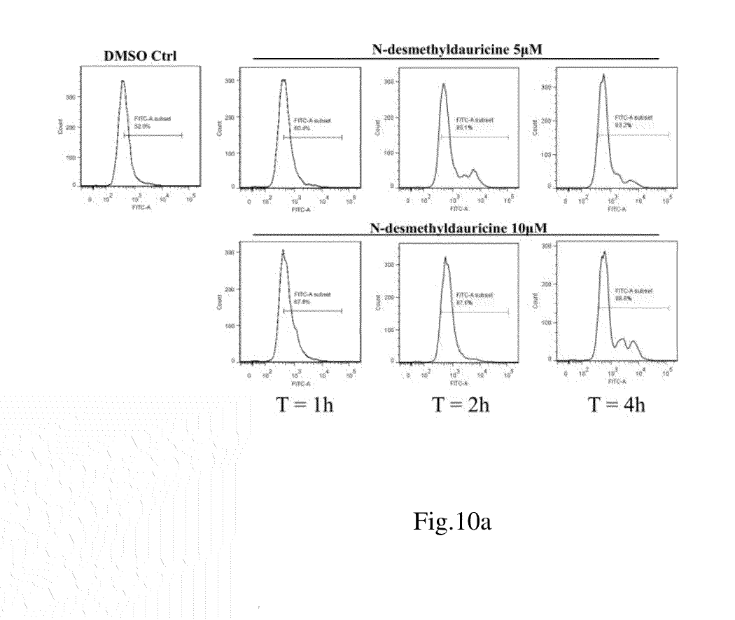

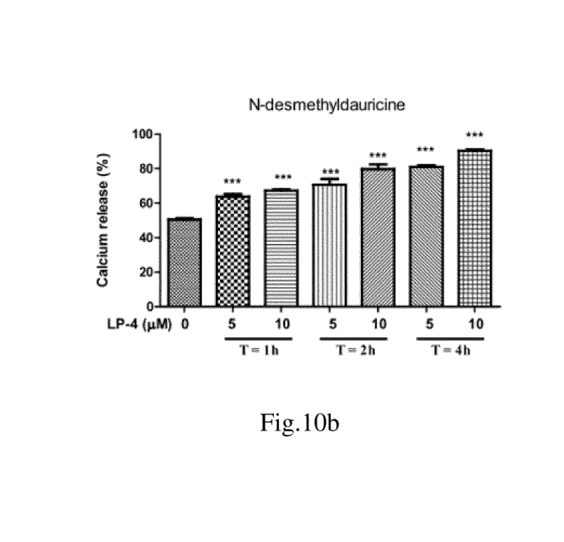

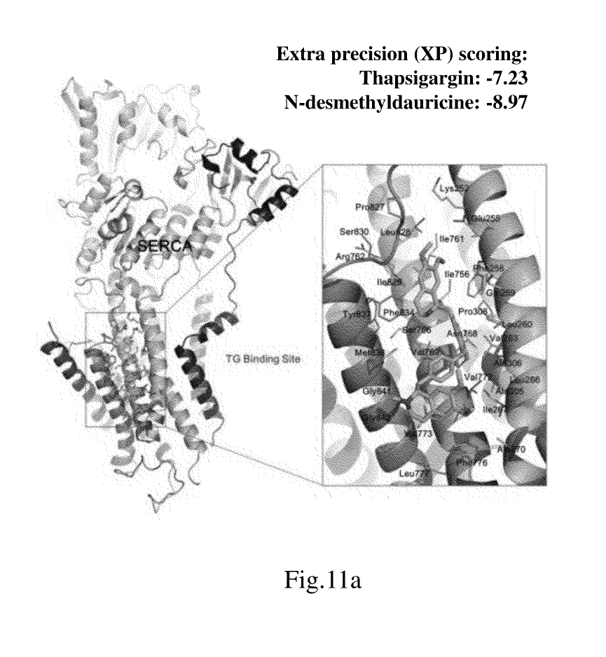

Results: N-desmethyldauricine was found to activate the phosphorylation of AMPK in a time dependent manner as shown in FIG. 8 and this activation was also accompanied by a concomitant reduction in its downstream p70S6K phosphorylation. In order to demonstrate whether the upstream of AMPK signaling is involved in N-desmethyldauricine-induced autophagy, specific inhibitors such as AMPK inhibitor, compound C; CaMKK-β inhibitor, STO-609; and calcium chelator, BAPTA/AM were used in the study. Results showed that there was a significant reduction in N-desmethyldauricine-induced GFP-LC3 puncta formation in HeLa cells treated with the presence of AMPK inhibitor (compound C), CaMKK-β inhibitor, STO-609, and calcium chelator, BAPTA/AM (BM) (as shown in FIG. 9a), in which findings were coincided with the LC3 conversion from LC3-I to LC3-II as shown in FIG. 9b. Given that calcium mobilization in cells will contribute to autophagy induction, this example further demonstrated that N-desmethyldauricine could be able to increase the cytosolic calcium level in time and dose dependent manner as shown in FIG. 10a and FIG. 10b.

### Conclusion

N-desmethyldauricine induces autophagy via mobilization of calcium signaling, leading to modulation of AMPK-mTOR signaling pathway.

### Example 8

This example describes an in vitro study to demonstrate the computational docking prediction and validation of SERCA as the direct target of N-desmethyldauricine during autophagy induction.

### Molecular Computational Docking

The 3D structure of N-desmethyldauricine was obtained from the PubChem (http://pubchem.ncbi.nlm.nih.gov). Then, the compound was preprocessed by the LigPrep¹⁹ which uses OPLS-2005 force field ²⁰ to obtain the corresponding low energy 3D conformers. The ionized state was assigned by using Epik3 at a target pH value of 7.0±2.0. The 3D crystal structure of the sarco(endo)plasmic reticulum Ca²⁺ ATPase (SERCA) was used in molecular docking. The 3D structure of SERCA was retrieved from the Protein Data Bank (PDB ID code 2AGV)²¹. The Protein Preparation Wizard was used to remove crystallographic water molecules, add hydrogen atoms, and assign partial charges based on OPLS-2005 force field²². Energy minimization was also performed and terminated when the root-mean-square deviation (RMSD) reached a maximum value of 0.3 Å. N-desmethyldauricine was docked into the thapsigargin (TG) binding site of the SERCA using Glide program²³ with the extra precision (XP) scoring mode. The docking grid box was defined by centering on TG in the SERCA.

### Measurement of SERCA Activity

Purified Ca²⁺ ATPase (SERCA1A) is prepared from female rabbit hind leg muscle²⁴. ATPase activity is determined using the enzyme-coupled method utilizing pyruvate kinase and lactate dehydrogenase as previously described in Michelangeli et al. (1990)²⁵. All SERCA inhibition data is fitted to the allosteric dose versus effect equation using Fig P (Biosoft):

Activity=minimum activity+(maximum activity−minimum activity)/(1+([I]IC₅₀)P).

### Results

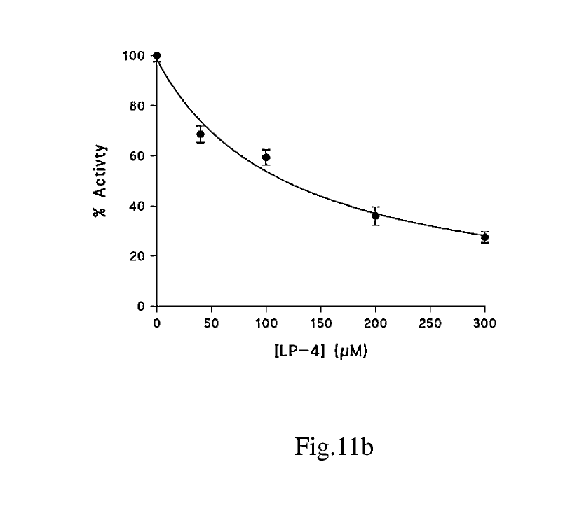

In molecular docking, 5000 poses were generated during the initial phase of the docking calculation, out of which the best 1000 poses were chosen for energy minimization by 1000 steps of conjugate gradient minimizations. The performance of molecular docking was evaluated by comparing the docked pose with the experimental structure for N-desmethyldauricine in the X-ray co-crystallized complex. TG in the X-ray co-crystallized complexes was re-docked into the binding sites and the RMSD for re-docked result of TG is 1.78 Å. Comparison of the docking pose of N-desmethyldauricine (XP score: −8.97) with the known SERCA inhibitor thapsigargin (XP score: −7.23) indicates that the two compounds were located in the space within the SERCA binding pocket as shown in FIG. 11a. To ascertain whether the SERCA pump is suppressed by N-desmethyldauricine, SERCA inhibitory effect is quantified using purified rabbit skeletal muscle sarcoplasmic reticulum (SR) membranes to measure the expression of the SERCA1A isoform by the SR membrances²⁶. Most of the existing SERCA inhibitors show similar inhibitory effect in SERCA isoform ^{15,16}. The SERCA1A pump (from rabbit skeletal muscle SR) is inhibited by N-desmethyldauricine in a dose-dependent manner (FIG. 11b), which is fitted to an allosteric dose versus effect equation.

### Conclusion

N-desmethyldauricine is confirmed to bind and suppress the SERCA, leading to the release of cytosolic calcium in cells.

### Example 9

This example describes an in vitro study to demonstrate that N-desmethyldauricine induce autophagic cell death in cells.

### Cell Culture and Flow Cytometry Analysis

Cell viability was measured using an annexin V staining kit (BD Biosciences, San Jose, Calif., USA). Briefly, Atg7 wild-type (Atg7^{+/+} or Atg7-wt) and Atg7 deficient (Atg7^{-/-} or Atg7-ko) mouse embryonic fibroblasts (MEFs) were treated with the 10 μM N-desmethyldauricine for 24 h. Cells were then harvested and analysed by multiparametric flow cytometry using FITC-Annexin V and Propidium iodide staining (BD Biosciences, San Jose, Calif., USA) according to the manufacturer's instructions. Flow cytometry was then carried out using a FACSCalibur flow cytometer (BD Biosciences, San Jose, Calif., USA). Data acquisition and analysis was performed with CellQuest (BD Biosciences, San Jose, Calif., USA). Data were obtained from three independent experiments.

### Results

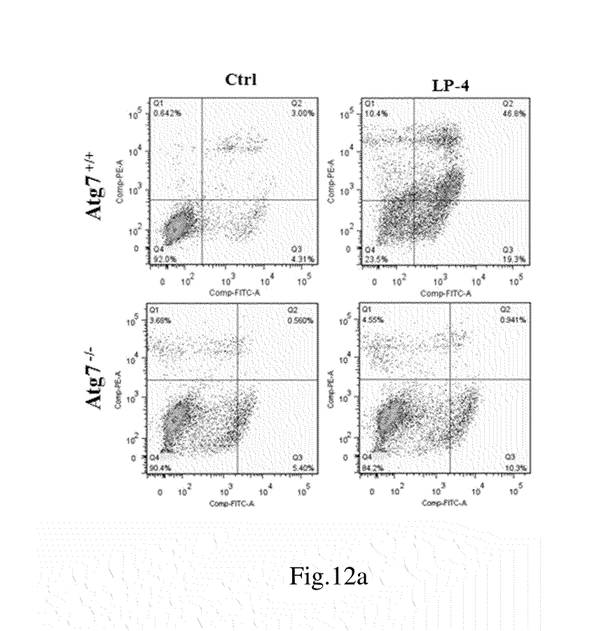

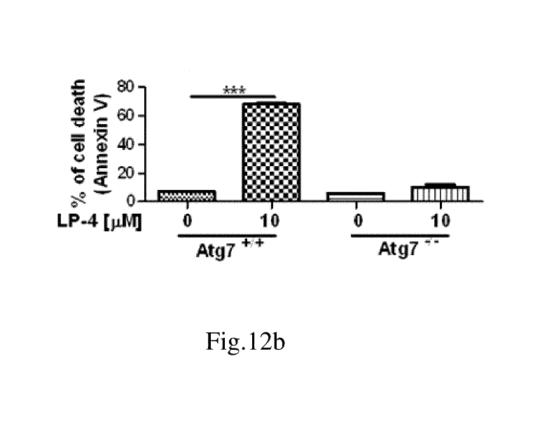

As shown in FIG. 12a and FIG. 12b, N-desmethyldauricine was found to markedly induce cell death in Atg7^{+/+} cells, but not in autophagy deficient cells (Atg7^{-/-})

### Conclusion

These findings suggest that N-desmethyldauricine-mediated cell death is autophagy dependent; in other words, N-desmethyldauricine is able to induce autophagic cell death.

### Example 10

This example describes an in vitro study to demonstrate that N-desmethyldauricine potently induces cell cytotoxicity in apoptosis-resistant cells.

### Cell Culture and Cytotoxicity Assay

The test compound of N-desmethyldauricine was dissolved in DMSO at a final concentration of 100 mmol/L and stored at −20° C. Cytotoxicity was assessed using the 3-(4,5-dimethylthiazol-2-yl)-2,5-diphenyltetrazolium bromide assay as previously described¹⁷. 2500 of caspase wild-type (caspase WT), caspase-3 deficient (caspase 3KO), caspase-7 deficient (caspase 7KO), caspase-3/-7 deficient (caspase 3/7 DKO), caspase-8 deficient (caspase 8KO), Bax-Bak wild-type (Bak-Bak WT) and Bax-Bak double knock out (Bak-Bak DKO) mouse embryonic fibroblasts (MEFs) were seeded on 96-well plates per well. After overnight pre-incubation, the cells were exposed to different concentrations of N-desmethyldauricine (namely 100, 50, 25, 12.5, 6.25, 3.125, 1.5625, 0.78, 0.39, 0.195, 0.079, 0.039 μmol) for 3 days. Subsequently, 10 μL of MTT reagents was added to each well and incubated at 37° C. for 4 hours, followed by the addition of 100 μL solubilization buffer (10% SDS in 0.01 mol/L HCl) and overnight incubation. Absorbance at 585 nm was determined from each well on the following day. The percentage of cell viability was calculated using the following formula: Cell viability (%)=Cells number treated/Cells number DMSO control×100. Data was obtained from three independent experiments.

### Results

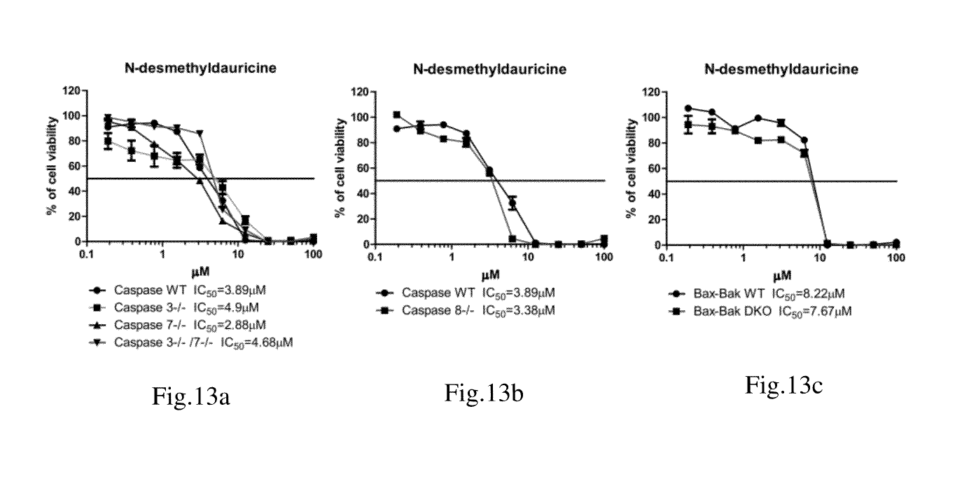

N-desmethyldauricine was found to exhibit similar cytotoxic effect on both wild-type and apoptosis-resistant cells, i.e. caspase-3/-7/-8 as compared to the caspase wild-type MEFs as shown in FIG. 13a, FIG. 13b and FIG. 13c. In addition, it also shows similar cytotoxicity in Bax-Bak DKO apoptosis-resistant cells as compared to Bax-Bak wild-type MEFs (also shown in FIG. 13a, FIG. 13b and FIG. 13c), indicating that N-desmethyldauricine is able to induce cell death in apoptosis-resistant cells.

### Conclusion

These findings suggest that N-desmethyldauricine is capable of inducing cell cytotoxicity in apoptosis-resistant cancer cells.

### Example 11

This example describes an in vitro study to demonstrate the clearance of mutant huntingtin and reduction of aggregates-mediated cytotoxicity by N-desmethyldauricine.

### Cell Culture and Cytotoxicity Assay

For cell viability assay measured by crystal violet staining, PC-12 cells were incubated in 35 mm disc followed by the addition of N-desmethyldauricine at 7.5 μM for 24 hours. The cells were then incubated with crystal violet for 10 minutes followed by a ddH₂O wash. The stained cells image was captured by CCD digital camera Spot RT3™ under the Nikon ECLIPSE 80i microscope with 4× magnification. Cell viability was quantified by dissolving stained cells in 10% acetic acid (200 μL/well). The colorimetric reading of the solute mixture was then determined by spectrophotometer at OD 560 nm. The percentage of cell viability was calculated using the following formula: Cell viability (%)=Cells number_{treated}/Cells number_{DMSO control}×100. Data was obtained from three independent experiments.

### Removal of Mutant Huntingtin

PC 12 cells were transfected transiently with EGFP-HDQ23/55/74 (Q23, Q55, Q74) plasmids for 24 hours using Lipofectamine Plus LTX reagent (Invitrogen) according to the manufacturer's protocol. The transfected cells were then treated with N-desmethyldauricine for 24 hours. The removal of mutant huntingtin, (Q23, Q55, Q74) were then quantitated by immunoblotting with antibody against EGFP.

### Results

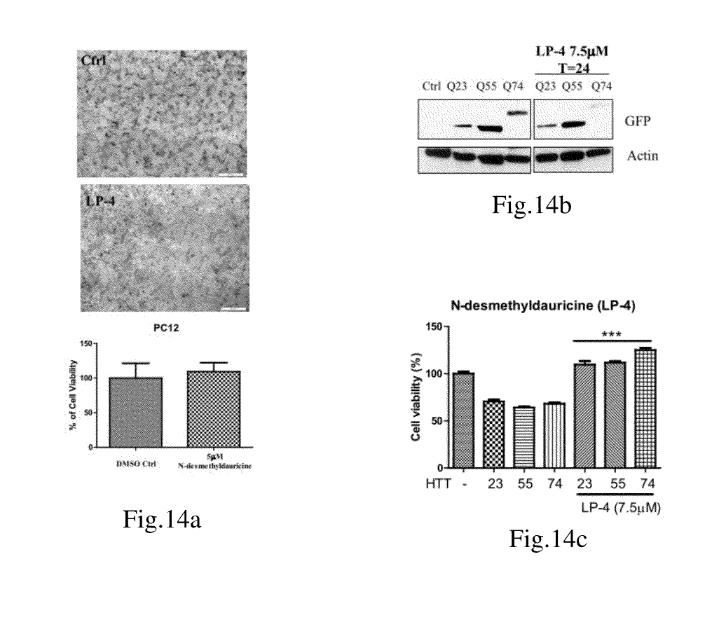

N-desmethyldauricine exhibited no toxicity in PC 12 cells at 7.5 μM as illustrated in FIG. 14a. In addition, 7.5 μM of N-desmethyldauricine is shown to enhance the clearance of overexpressed EGFP-tagged mutant huntingtin (Q23, Q55, Q74) with 23, 55 and 74 CAG repeats as measured by immunoblotting against EGFP antibody as shown in FIG. 14b. On the other hand, addition of 7.5 μM of N-desmethyldauricine would reduce the huntingtin aggregates-mediated cytotoxicity and enhance the cell viability of the mutant huntingtin overexpressed PC 12 cells as illustrated in FIG. 14c. By using the neuronal cells PC12, LP-4 was found to be able to remove the Huntingtin CAG repeats Q23, Q55 and Q74, which showed that N-desmethyldauricine is a neuro-protective agent against Huntington's disease.

### Conclusion

N-desmethyldauricine is shown to work as a novel neuroprotective agent through accelerating the clearance of mutant huntingtin and reduce the cell cytotoxicity of huntingtin aggregates.

### SUMMARY

This invention covers the anti-cancer effect of N-desmethyldauricine. In one embodiment, the anti-cancer effect is made possible through the induction of autophagic cell death in a panel of cancer cells and apoptosis-resistant cells. In addition, the invention further covers the neuroprotective effect of N-desmethyldauricine on neuronal cells via enhancing the clearance of mutant huntingtin and reducing its mediated cell cytotoxicity.

In another embodiment, this invention provides that, N-desmethyldauricine exhibits specific cytotoxic effect toward human cancer cells. N-desmethyldauricine is capable to induce autophagy in a panel of cancer and normal cells, and animals; induce autophagosomes/autolysosomes formation in cells and animals; induce autophagic protein LC3 conversion in cells and animals; induce autophagy in Atg7 dependent manner; induce autophagy via regulation of genes, i.e. Ulk1 and PERK; induce autophagy via mobilization of calcium signaling and modulation of AMPK-mTOR signaling pathway; induce autophagy via inhibition of SERCA, thereby mobilizing calcium signaling and modulate AMPK-mTOR signaling pathway; and induce autophagic cell death mechanism in Atg7 containing cancer cells. N-desmethyldauricine exhibits potent cytotoxic effect towards apoptosis-resistant cancer cells. N-desmethyldauricine is capable to enhance the clearance of mutant huntingtin and reduce the mutant huntingtin aggregates-mediated cell cytotoxicity. N-desmethyldauricine can be developed as novel anti-cancer and neuroprotective agents for patients with cancers or neurodegenerative diseases.

In this invention, it is the first report that an alkaloid compound, N-desmethyldauricine induces autophagy in a panel of cancer cells and apoptosis-resistant cells. Mechanistic studies revealed that N-desmethyldauricine-induced autophagy occurred by direct inhibition of sarcoplasmic/endoplasmic reticulum Ca²⁺-ATPase (SERCA), leading to the increase of intracellular calcium ion levels and activating the ULK-1-CaMKK-β-AMPK-mTOR signaling cascade. The activation of these pathways ultimately leads to autophagy related cell death in both cancer cells and apoptosis-resistant cells. On the other hand, N-desmethyldauricine is capable to promote the degradation of mutant huntingtin with 23, 55 and 74 CAG repeats in PC12 cells via autophagy induction. Taken together, this invention provides novel insights into the autophagic effect of N-desmethyldauricine and evaluates its potential use in anti-cancer or neurodegenerative diseases in future.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### REFERENCES

1. Levine, B. & Kroemer, G. Autophagy in the pathogenesis of disease. Cell 132, 27-42 (2008).
2. Pallauf, K. & Rimbach, G. Autophagy, polyphenols and healthy ageing. Ageing Res Rev 12, 237-252.
3. Rubinsztein, D. C., Gestwicki, J. E., Murphy, L. O. & Klionsky, D. J. Potential therapeutic applications of autophagy. Nat Rev Drug Discov 6, 304-312 (2007).
4. Ravikumar, B., Duden, R. & Rubinsztein, D. C. Aggregate-prone proteins with polyglutamine and polyalanine expansions are degraded by autophagy. Hum Mol Genet 11, 1107-1117 (2002).
5. Ravikumar, B., et al. Inhibition of mTOR induces autophagy and reduces toxicity of polyglutamine expansions in fly and mouse models of Huntington disease. Nat Genet 36, 585-595 (2004).
6. Webb, J. L., Ravikumar, B., Atkins, J., Skepper, J. N. & Rubinsztein, D. C. Alpha-Synuclein is degraded by both autophagy and the proteasome. J Biol Chem 278, 25009-25013 (2003).
7. Rubinsztein, D. C., Marino, G. & Kroemer, G. Autophagy and aging. Cell 146, 682-695.
8. Sarkar, S., et al. Small molecules enhance autophagy and reduce toxicity in Huntington's disease models. Nat Chem Biol 3, 331-338 (2007).
9. Berger, Z., et al. Rapamycin alleviates toxicity of different aggregate-prone proteins. Hum Mol Genet 15, 433-442 (2006).
10. Ravikumar, B., et al. Regulation of mammalian autophagy in physiology and pathophysiology. Physiol Rev 90, 1383-1435.
11. Mathew, R., et al. Autophagy suppresses tumor progression by limiting chromosomal instability. Genes Dev 21, 1367-1381 (2007).
12. Liang, X. H., et al. Induction of autophagy and inhibition of tumorigenesis by beclin 1. Nature 402, 672-676 (1999).
13. Kondo, Y., Kanzawa, T., Sawaya, R. & Kondo, S. The role of autophagy in cancer development and response to therapy. Nat Rev Cancer 5, 726-734 (2005).
14. Hoyer-Hansen, M., Bastholm, L., Mathiasen, I. S., Elling, F. & Jaattela, M. Vitamin D analog EB1089 triggers dramatic lysosomal changes and Beclin 1-mediated autophagic cell death. Cell Death Differ 12, 1297-1309 (2005).
15. Law, B. Y., et al. Alisol B, a novel inhibitor of the sarcoplasmic/endoplasmic reticulum Ca(2+) ATPase pump, induces autophagy, endoplasmic reticulum stress, and apoptosis. Mol Cancer Ther 9, 718-730.
16. Wong, V. K., et al. Saikosaponin-d, a novel SERCA inhibitor, induces autophagic cell death in apoptosis-defective cells. Cell Death Dis 4, e720.
17. Wong, V. K., Zhou, H., Cheung, S. S., Li, T. & Liu, L. Mechanistic study of saikosaponin-d (Ssd) on suppression of murine T lymphocyte activation. J Cell Biochem 107, 303-315 (2009).
18. Liu, M. J., Wang, Z., Ju, Y., Wong, R. N. & Wu, Q. Y. Diosgenin induces cell cycle arrest and apoptosis in human leukemia K562 cells with the disruption of Ca2+ homeostasis. Cancer Chemother Pharmacol 55, 79-90 (2005).
19. Schrödinger, L., New York, N.Y. LigPrep, version 2.3. (2009).
20. Kaminski, G. A. F., R. A.; Tirado-Rives, J.; Jorgensen, W. L. Evaluation and reparametrization of the OPLS-AA force field for proteins via comparison with accurate quantum chemical calculations on peptides. J. Phys. Chem. B 105, 6474-6487 (2001).
21. Obara, K., et al. Structural role of countertransport revealed in Ca(2+) pump crystal structure in the absence of Ca(2+). Proc Natl Acad Sci U S A 102, 14489-14496 (2005).
22. Epik, version 2.0, Schrodinger, LLC, New York, N.Y,. (2009).
23. Glide, version 5.5, Schrodinger, LLC, New York, N.Y. (2009).
24. Michelangeli, F. & Munkonge, F. M. Methods of reconstitution of the purified sarcoplasmic reticulum (Ca(2+)-Mg2+)-ATPase using bile salt detergents to form membranes of defined lipid to protein ratios or sealed vesicles. Anal Biochem 194, 231-236 (1991).
25. Michelangeli, F., Colyer, J., East, J. M. & Lee, A. G. Effect of pH on the activity of the Ca2++Mg2(+)-activated ATPase of sarcoplasmic reticulum. Biochem J 267, 423-429 (1990).
26. Wu, K. D., Lee, W. S., Wey, J., Bungard, D. & Lytton, J. Localization and quantification of endoplasmic reticulum Ca(2+)-ATPase isoform transcripts. Am J Physiol 269, C775-784 (1995).

What is claimed is:

1. A method of treating cancer comprising administering a therapeutically effective amount of N-desmethyldauricine to a subject in need thereof.

2. The method of claim 1, wherein said cancer is selected from the group consisting of cervical cancer, lung cancer, breast cancer, prostate cancer and liver cancer.

3. The method of claim 1, wherein said N-desmethyldauricine selectively induces autophagic cell death in cancer cells or apoptosis-resistant cells via direct inhibition of SERCA.

4. A method of treating neurodegenerative disorder comprising administering a therapeutically effective amount of N-desmethyldauricine to a subject in need thereof.

5. The method of claim 4, wherein said N-desmethyldauricine removes Huntingtin aggregates via autophagy induction and reduces the aggregate-mediated cell cytotoxicity in neuronal cells.

6. The method of claim 4, wherein said neurodegenerative disease is selected from a group of consisting of Alzheimer's disease, Parkinson's disease, Huntington's disease, amyotrophic lateral sclerosis, ataxia telangiectasia, spinocerebellar atrophy and multiple sclerosis.
